(19) United States
(12) Patent Application Publication
(10) Pub. No.: US 2017/0007704 A1
(43) Pub. Date: Jan. 12, 2017

(54) CARRIER AND PHARMACEUTICAL COMPOSITIONS FOR INTRASINAL DELIVERY AND USES THEREOF

(71) Applicant: David Ram, Nanaimo (CA)

(72) Inventor: David Ram, Nanaimo (CA)

(21) Appl. No.: 14/794,870

(22) Filed: Jul. 9, 2015

(51) Int. Cl. A61K 47/34; A61K 31/65; A61K 31/7048; A61K 31/546; A61K 9/00; A61K 31/496; A61K 31/505; A61K 31/635; A61K 47/14; A61K 47/10; A61K 31/58; A61K 31/7036

(52) U.S. Cl. CPC A61K 47/34; A61K 31/58; A61K 31/65; A61K 31/7048; A61K 31/546; A61K 31/7036; A61K 31/496; A61K 31/505; A61K 31/635; A61K 47/14; A61K 47/10; A61K 9/0043

(57) ABSTRACT

Disclosed are carrier compositions for local administration of one or more active pharmaceutical ingredients to a subject in need of treatment. The carrier compositions comprise an in situ gelling agent comprising a poloxamer, particularly, an in situ gelling agent comprising Poloxamer 407, a biofilm inhibiting agent and a mucosal lubricant. Further disclosed are pharmaceutical compositions comprising the carrier composition and uses thereof for the treatment of chronic rhinosinutitus.

### FIELD OF INVENTION

The present invention relates to a carrier composition for the delivery of one or more pharmaceutical actives into the paranasal sinuses of a subject and uses thereof. More particularly, the present invention relates to a carrier composition comprising an in situ gelling agent and uses thereof in the treatment of chronic rhinosinusitis.

### BACKGROUND

Chronic rhinosinusitis is a group of disorders characterized by inflammation of the nose and paranasal sinuses. The pathophysiology of chronic rhinosinusitis is believed to arise primarily from the persistent inflammation of the mucosa of the nose and paranasal sinuses, and perhaps the underlying bone. Symptoms of the condition include purulent nasal discharge, postnasal drip, nasal obstruction, facial congestion/pressure/fullness, facial pain and hyposmia.

There are various predisposing factors for the condition including environmental factors (e.g. pollution, allergens, viruses, bacteria and moulds), general host factors (e.g. genetic, granulomatous disorders, immune deficiency, cystic fibrosis, and ciliary defects), and local host factors (e.g. chronic localized inflammation, anatomic obstruction, polyps and tumours).

The etiology of chronic rhinosinusitis is unclear. Causative factors may include bacterial colonization, fungal colonization, bacterial biofilms, eosinophilic, neutrophilc and lymophocytic infiltration, up-regulation of Th-2 associated cytokines and tissue remodeling. Various bacterial pathogens are associated with the condition, including S. aureus, S. pneumonia, S. pyrogenes, H. influenzae, M. catarrhalis, Enterobacteriaceae and other various anaerobic species. S. aureus and Enterobacteriaceae are more commonly associated with chronic rhinosinusitis as compared to acute rhinosinusitis.

Treatment of chronic rhinosinusitis is aimed at reducing mucosal inflammation, controlling infection, and restoring mucociliary clearance within the paranasal sinuses. Pharmacotherapy in conjunction with non-pharmacological measures is generally recommended (e.g. environmental control, saline nasal/sinus rinses). In some refractory cases, surgical intervention may be required.

Pharmacotherapeutic regimens often include the use of oral or topical corticosteroids to control inflammation in conjunction with an oral or topical antibiotic or antifungal to control the underlying microbial infection. Other medications such as decongestants, antihistamines or leukotriene inhibitors may be indicated in some cases.

Despite the variety of treatment options, chronic rhinosinusitis is a frustrating and difficult condition to treat. The interplay between the pathogenesis and inflammatory stimuli is complex and not fully understood. Further, current treatment regimens require the long-term use of pharmaceuticals, with many drugs requiring treatment regimens of weeks and months to achieve adequate systemic concentrations to be effective. Sustained concentrations of active pharmaceutical compounds often leads to intolerable adverse effects that affect patient outcomes and patient compliance.

Locally administered pharmaceuticals, including topical formulations, are useful to avoid the complications with long-term systemic administration of pharmaceuticals. Conventional therapies for chronic rhinosinusitis include the use of topical corticosteroids, topical antibiotics or antifungals.

Topical corticosteroids are considered first-line therapy for the treatment of chronic rhinosinusitis and have been shown to reduce sinus and nasal inflammation and associated symptoms. These medications may be used on their own or in conjunction with an oral medication used to combat the underlying pathogen(s). Unlike long-term systemic use of corticosteroids, topical corticosteroids, which are administered in the form of a nasal spray, are associated with less adverse effects. However, adverse effects including nasal irritation, mucosal bleeding, dryness and crusting are common with topical corticosteroids.

Topical antibiotics are a further treatment option. Antibiotics administered via nebulizer over a period of 3-weeks have been shown to reduce facial pain/pressure, nasal discharge and have resulted in longer infection-free periods. While this remains a reasonable treatment option, additional investigations into optimal dosing, treatment durations and delivery mechanisms continue to be pursued.

While topical therapies provide an effective treatment option whether used alone or in conjunction with systemic and/or non-pharmacological treatments, there are inadequacies with current topical treatments. Commercially available topical corticosteroids and antibiotics are only available as nasal sprays, which provide limited contact between the administered pharmaceutical active and the mucosa of the paranasal sinuses. By their nature, corticosteroid nasal sprays require frequent administration, raising the potential of sub-optimal patient compliance. Further, biofilms present in the paranasal sinuses can act as a barrier, further limiting contact between the administered pharmaceutical active and the mucosa of the paranasal sinuses.

### SUMMARY OF INVENTION

In one aspect, disclosed is a carrier composition.

In another aspect, disclosed is a pharmaceutical composition, wherein the pharmaceutical composition comprises at least one active pharmaceutical ingredient and a carrier composition.

In a further aspect, disclosed is a method for delivering of one or more active pharmaceutical ingredients to at least one paranasal sinus of a subject in need of treatment, the method comprising: incorporating the at least one active pharmaceutical ingredient into a carrier composition to provide a liquid formulation and administering the liquid formulation into the paranasal sinus of the subject; wherein upon contact of the liquid formulation with the mucosa of the paranasal sinus, the liquid formulation forms an in situ gel.

In one embodiment of the disclosed carrier composition, the disclosed pharmaceutical composition or the disclosed method, the carrier composition comprises an in situ gelling agent, a biofilm inhibiting agent, and a mucosal lubricant, wherein the in situ gelling agent comprises a poloxamer.

In a further embodiment of the disclosed carrier composition, the disclosed pharmaceutical composition or the disclosed method, the in situ gelling agent comprises Poloxamer 407. The carrier composition may comprise about 20% w/v of the Poloxamer 407.

In a further embodiment of the disclosed carrier composition, the disclosed pharmaceutical composition or the disclosed method, the mucosal lubricant comprises caprylic triglycerides. The carrier composition may comprise about 5% to about 15% v/v of the caprylic triglycerides.

In a further embodiment of the disclosed carrier composition, the disclosed pharmaceutical composition or the disclosed method, the biofilm inhibiting agent is xylitol. The carrier composition may comprise about 1% to about 3% w/v of the xylitol.

In a further embodiment of the disclosed carrier composition, the disclosed pharmaceutical composition or the disclosed method, the carrier composition comprises about 20% w/v of the Poloxamer 407, about 5% to about 15% v/v of the caprylic triglycerides and about 1% to about 3% w/v of the xylitol.

In a further embodiment of the disclosed carrier composition, the disclosed pharmaceutical composition or the disclosed method, the carrier composition comprises about 20% w/v of the Poloxamer 407, about 5-15% v/v of the caprylic triglycerides and about 1% w/v of the xylitol.

In a further embodiment of the disclosed pharmaceutical composition or the disclosed method, the active pharmaceutical ingredient is an antibiotic, an antifungal, an antihistamine, a corticosteroid, a decongestant, an anesthetic, a prostaglandin, or a vitamin.

In a further embodiment of the disclosed pharmaceutical composition or the disclosed method, the antibiotic is an aminoglycoside, a carbapenem, a cephalosporin, a fluoroquinolone, a dihydrofolate reductase inhibitor, a glycopeptide, a penicillin, a polymyxin, a sulfonamide, a tetracycline or the pharmaceutically acceptable salts or esters thereof.

In a further embodiment of the disclosed pharmaceutical composition or the disclosed method, the antifungal is itraconazonle, amphotericin B, ketoconazole, fluconazole, micafungin, capsofungin or the pharmaceutically acceptable salts or esters thereof.

In a further embodiment of the disclosed pharmaceutical composition or the disclosed method, the antihistamine is diphenhydramine or its pharmaceutically acceptable salts or esters thereof.

In a further embodiment of the disclosed pharmaceutical composition or the disclosed method, the anesthetic is lidocaine, ketamine or the pharmaceutically acceptable salts or esters thereof.

In a further embodiment of the disclosed pharmaceutical composition or the disclosed method, the corticosteroid is budesonide, beclomethasone, ciclesonide, fluticasone, triamcinolone or the pharmaceutically acceptable salts or esters thereof.

In a further embodiment of the disclosed pharmaceutical composition or the disclosed method, the prostaglandin is misoprostol or its pharmaceutically acceptable salts or esters.

In a further embodiment of the disclosed pharmaceutical composition or the disclosed method, the vitamin is ergocalciferol, cholecalciferol or the pharmaceutically acceptable salts or esters thereof.

In a further embodiment of the disclosed pharmaceutical composition or the disclosed method, the active pharmaceutical ingredient is phenytoin or its pharmaceutically acceptable salts or esters.

In a further embodiment of the disclosed pharmaceutical composition or the disclosed method, the active pharmaceutical ingredient is tranilast or its pharmaceutically acceptable salts or esters.

In a further embodiment of the disclosed pharmaceutical composition or the disclosed method, the active pharmaceutical ingredient is sodium chromoglycate or its pharmaceutically acceptable salts or esters.

In a further embodiment of the disclosed pharmaceutical composition or the disclosed method, the active pharmaceutical ingredient is arginine or its pharmaceutically acceptable salts or esters.

In a further embodiment of the disclosed pharmaceutical composition or the disclosed method, the active pharmaceutical ingredient is fluorescein or its pharmaceutically acceptable salts or ester.

In an embodiment of the disclosed method, the liquid formulation is administered into the paranasal sinus of the subject using a syringe connected to a cannula, the cannula being inserted into a sinus cavity, turbinate and/or meatus.

In a further embodiment of the disclosed method, the subject is in need of treatment for chronic rhinosinusitis.

### DESCRIPTION

It has now surprisingly been found that local administration of pharmaceutical active(s) within the paranasal sinuses together with a carrier composition comprising an in situ gelling agent, a biofilm inhibitor and a mucosal lubricant as described herein, provides improved delivery of the pharmaceutical actives and improved treatment outcomes in subjects with chronic rhinosinusitis.

Without being bound to a particular theory, it is believed that use of the carrier composition improves delivery of the pharmaceutical actives(s) by increasing the residence time of the pharmaceutical active(s) in the paranasal sinuses thereby prolonging the localized release of the pharmaceutical active(s). It is believed that the residence time of the pharmaceutical active(s) is increased through the formation of an in situ gel containing the pharmaceutical active(s) onto the sinus mucosa. As compared to prior art formulations, use of the carrier composition may provide residence times in the order of days. This is in sharp contrast to the relatively short residence time of prior art formulations, such as those for nasal spray administration, which may be only minutes or hours.

By using the carrier composition described herein, locally administered pharmaceutical active(s) may remain in extended contact with the sinus mucosa (e.g. 4-7 days, depending on the type of pharmaceutical active) thereby reducing the need for frequent administration or extended treatment regimens. By decreasing the complexity and/or length of the treatment, patient compliance may be improved. Further, systemic administration, such as intravenous administration requiring hospitalization, may be avoided. Consequently, the adverse effects caused by prolonged systemic concentrations of active pharmaceutical ingredients may also be avoided.

It is further believed that delivery of the pharmaceutical active(s) to the paranasal sinuses is improved due to enhanced biofilm penetration of the pharmaceutical active(s). Use of the carrier composition described herein, which includes a biofilm inhibiting agent, may improve penetration of the pharmaceutical active(s) through biofilms present in the sinuses, thereby increasing the amount of pharmaceutical actives delivered onto the sinus mucosa.

The use of the carrier composition for the local administration of pharmaceutical active(s) for the treatment of chronic rhinosinusitis has surprisingly been found to provide improved treatment outcomes as compared to conventional treatment methods in which the pharmaceutical actives are administered locally (e.g. as a nasal spray) or administered systemically (e.g. intravenous administration). As described in further detail in the Examples, patients treated using a variety of pharmaceutical actives locally administered using the carrier composition described herein were surprisingly found to have improved treatment outcomes including the resolution of symptoms and infections that had been nonresponsive to previous conventional treatment methods. Further, the carrier compositions described herein were found to provide additional therapeutic benefits including soothing dry tissue in the sinuses, a common problem for patients with sinus disease.

### Carrier Composition

As used herein an “active pharmaceutical ingredient” refers to any substance that provides pharmacological activity or other direct effect in the diagnosis, cure, mitigation, treatment or prevention of disease or affects the function of a subject's physiology. The term “subject” as used herein refers to a mammal, preferably a human. The term “paranasal sinus” as used herein in reference to a human subject, includes the maxillary sinuses, the frontal sinuses, the ethmoid sinuses and the sphenoidal sinuses.

In one aspect, disclosed is a carrier composition for use in the local administration of one or more active pharmaceutical ingredients to the paranasal sinus(es) of a subject in need of treatment. The carrier composition may comprise an in-situ gelling agent, a biofilm inhibitor and a mucosal lubricant. Other possible components include non-irritating pH adjustors such as sodium phosphate dibasic and sodium phosphate monobasic, non-irritating antimicrobial preservatives such as imidurea and sodium dehydroacetate, and probiotics such as Lactobacillus Acidophilus DDS-1.

### In-Situ Gelling Agent

As used herein an “in situ gelling agent” refers to a composition that is liquid at lower temperatures (e.g. under refrigeration at about 2-10° C.) and converts into a gel at mammalian physiologic temperatures (e.g. ca. 37° C. for humans). As a result of the inclusion of the in situ gelling agent, the carrier composition can be administered as a liquid that forms a thermo-reversible in situ gel when the carrier composition comes into contact with the sinus mucosa of the subject being treated.

The carrier composition may comprise an aqueous solution of a poloxamer and more preferably, Poloxamer 407, as the in situ gelling agent. The choice of poloxamer and the amount used are selected based on the desired viscosity of the resulting carrier composition. The carrier composition when converted into gel form in the paranasal sinuses should have a viscosity that promotes adherence of the in situ gel on the sinus mucosa. The carrier composition may comprise about 5% to about 50% w/v of the poloxamer, preferably about 10% to about 20% w/v of the poloxamer; and more preferably about 20% w/v of the poloxamer.

In a preferred embodiment, the carrier composition comprises an aqueous solution of Poloxamer 407, wherein the amount of the Poloxamer 407 is about 20% w/v of the carrier composition. For example, the carrier composition can be prepared using PCCA® Poloxamer 407 20% Gel NF (PCCA, London, Canada), which is comprised of Poloxamer 407 NF (20% w/v), purified water, imidurea, sodium phosphate dibasic, sodium phosphate monobasic and sodium dehydroacetate.

### Biofilm Inhibiting Agent

The carrier composition may further comprise a biofilm inhibiting agent. As used herein, a “biofilm inhibiting agent” refers to a compound or composition that eliminates, in whole or part, an existing biofilm and/or a compound or composition that prevents or reduces the formation of a biofilm.

In use, following local administration of the carrier composition within the paranasal sinuses, the prolonged period of contact with the mucosa of the paranasal sinuses offered by the resulting in situ gel permits the biofilm inhibiting agent to reduce or eliminate the presence of biofilms in the paranasal sinus, for example, through the bacteriocidal or bacteriostatic properties of the biofilm inhibiting agent. The choice of the biofilm inhibiting agent will depend on the pathogens of the biofilm and the other components of the formulation of the invention. Examples of suitable biofilm inhibiting agents include but are not limited to xylitol, ribose (in combination with xylitol), edetate disodium, sodium ascorbate, sucralose, sorbitol, ambroxol, N-Acetylcysteine. The amount of biofilm inhibiting agent included in the carrier composition will depend on the choice of biofilm inhibiting agent. Generally, the carrier composition may comprise about 1 to 10% w/v of the biofilm inhibiting agent and more preferably about 1 to 5% w/v of the biofilm inhibiting agent.

In a preferred embodiment, the carrier composition comprises xylitol as the biofilm inhibiting agent. For example, the carrier composition can be prepared using PCCA® Xylitol NF (PCCA, London, Canada). The carrier composition may comprise about 1 to 5% w/v of xylitol, preferably about 1 to 3% w/v of xylitol and more preferably about 1% w/v of xylitol.

### Mucosal Lubricant/Dispersant

The carrier composition may further comprise a mucosal lubricant. As used herein, a “mucosal lubricant” is a compound or composition that soothes and/or protects mucosal tissue by promoting moisture retention on or within the tissue.

In a preferred embodiment, the mucosal lubricant comprises caprylic triglycerides, for example, PCCA® Caprylic Triglycerides (PCCA, London, Canada). As compared to wetting agents, such as propylene glycol, caprylic triglycerides do not cause mucosal irritation. Further, the caprylic triglycerides can function as a dispersing agent for non-water soluble or poorly water soluble components, for example, non-soluble active pharmaceutical ingredients when the carrier composition is used in the preparation of a pharmaceutical composition. The inclusion of caprylic triglycerides prevents or eliminates the clumping and/or sedimentation of non-water soluble or poorly water soluble components.

In most cases, it is desirable to include caprylic triglycerides in the carrier composition for their soothing mucosal lubricant properties regardless of the solubility of the other components of the carrier composition or the pharmaceutical composition prepared using the carrier composition. In a preferred embodiment, the carrier composition comprises about 5 to 20% v/v of caprylic triglycerides, preferably about 5 to 15% v/v caprylic triglycerides.

The carrier compositions can be prepared using conventional pharmaceutical manufacturing methods. Generally, an aqueous solution of the selected in situ gelling agent is prepared using the selected poloxamer and combined with the selected biofilm inhibitor and mucosal lubricant to form a uniform mixture. Water or another suitable aqueous carrier can be added to adjust the volume of the resulting mixture. If the carrier composition is intended to be used with one or more non-water soluble or poorly water soluble components, it is preferable to first combine the non-water soluble or poorly water soluble components with a dispersing agent, preferably caprylic triglycerides, prior to the addition to the other components.

### Pharmaceutical Compositions

In a further aspect, provided is a pharmaceutical composition for local administration to the paranasal sinus(es) of a subject in need of treatment. The pharmaceutical composition comprises one or more active pharmaceutical ingredients incorporated into a carrier composition. The carrier composition may comprise an in-situ gelling agent, a biofilm inhibitor and a mucosal lubricant as described above.

The pharmaceutical composition can be prepared using one or more active pharmaceutical ingredients. The selected active pharmaceutical ingredient may be provided as a pharmaceutically acceptable salt or ester. As used herein the term “acceptable” is in the sense of being compatible with the other ingredients of the pharmaceutical composition and not deleterious to the recipient thereof.

In a preferred embodiment, the pharmaceutical composition is for treating chronic rhinosinusitis. The pharmaceutical composition can be formulated using active pharmaceutical ingredients used in conventional methods for the treatment of chronic rhinosinusitis, such as but not limited to: antibiotics, antifungals, and corticosteroids. The pharmaceutical composition may comprise one or more different antibiotics, one or more different antifungals, one or more corticosteroids, or a combination thereof. The pharmaceutical composition can also be formulated with other active pharmaceutical ingredients for topical application to the paranasal sinuses including, but not limited to: antihistamines, decongestants, anesthetics, prostaglandins, vitamins or combinations thereof. Depending on the symptoms to be treated, these additional active pharmaceutical ingredients can be used as a separate treatment regimen or in combination with one or more different antibiotics, one or more different antifungals, and/or one or more corticosteroids.

Examples of antibiotics that may be included in the pharmaceutical composition include, but are not limited to the following classes of antibiotics: aminoglycosides, carbapenems, cephalosporins, fluoroquinolones, dihydrofolate reductase inhibitors, glycopeptides, penicillins, polymyxins, sulfonamides and tetracyclines. It will be appreciated that selection of the appropriate antibiotic will depend on the susceptibility of the pathogens present in the paranasal sinuses of the subject to be treated. The following exemplary antibiotics and concentrations can be used in the preparation of a typical single dosage of the pharmaceutical composition (total volume ca. 10 ml), and more preferably a single dosage form for the treatment of chronic rhinosinusitis:

gentamicin—80 mg to 160 mg/10 mL

tobramycin—80 mg to 160 mg/10 mL

sulfamethoxazole—400 mg to 800 mg/10 mL

trimethoprim—80 mg to 160 mg/10 mL

doxycycline—100 mg to 200 mg/10 mL

minocycline—100 mg to 200 mg/10 mL

ciprofloxacin—100 mg-200 mg/10 mL

vancomycin—500 mg to 1000 mg/10 mL

ampicillin—500 mg to 2000 mg/10 mL

colistimethate sodium—150 mg/10 mL

ceftazidime—5 mg/mL

imipenem—250 mg to 500 mg/10 mL.

Examples of corticosteroids that may be included in the pharmaceutical composition include, but are not limited to: budesonide, ciclesonide, clomethasone, dexamethasone, fluticasone, prednisone, prednisolone and triamcinolone. These corticosteroids when applied topically are potent anti-inflammatory agents and avoid significant systemic concentrations as they are locally administered. In a preferred embodiment for the treatment of chronic rhinosinusitis, the pharmaceutical composition may comprise budesonide at a concentration of 0.5 to 4 mg/10 mL.

The pharmaceutical composition may include one or more antifungals. The choice of antifungal will depend on the susceptibility of the pathogens present in paranasal sinuses to be treated. Preferred antifungals and their exemplary concentrations for a typical single dosage of the pharmaceutical composition (total volume ca. 10 ml), and more preferably a single dosage form for the treatment of chronic rhinosinusitis, include, but are not limited to:

itraconazole—100 mg to 200 mg/10 mL

amphoteracin B—3 mg to 10 mg/10 mL.

Ketoconazole—100 mg to 200 mg/10 ml

Fluconazole—100 mg to 400 mg/10 mL

Micafungin—10 mg to 50 mg/10 mL

Capsofungin—10 mg to 50 mg/10 mL

Other exemplary active pharmaceutical ingredients that can be used alone or in combination in the preparation of the pharmaceutical composition include, but are not limited to:

fluorescein—0.1-0.25% w/v

ketamine—1% w/v

lidocaine—1-4% w/v

misoprostol—0.0024% w/v

phenytoin—1-2% w/v

vitamin D (e.g. ergocalciferol, cholecalciferol)—1000 IU to 5000 IU/10 mL

arginine—0.5% w/v

sodium chromoglicate—0.5% w/v

diphenhydramine—1-2% w/v.

tranilast—100 mg-300 mg/10 mL

The pharmaceutical compositions can be prepared using conventional pharmaceutical manufacturing methods. Generally, an aqueous solution of the selected in situ gelling agent is prepared using the selected poloxamer and combined with the selected biofilm inhibitor, mucosal lubricant and one or more active pharmaceutical ingredients to form a uniform mixture. Water or another suitable aqueous carrier can be added to adjust the volume of the resulting mixture. Where the selected active pharmaceutical ingredient(s) is non-water soluble or poorly soluble, it is preferable to first combine the non-water soluble or poorly water soluble components with a dispersing agent, preferably caprylic triglycerides, prior to the addition to the other components.

In a preferred embodiment, the pharmaceutical compositions can be prepared on a patient by patient basis and single dosages provided in a syringe for administration. The pharmaceutical compositions can be prepared under sterile conditions in accordance with USP 797 Guidelines.

In embodiments where all the components of the formulation are water soluble, the pharmaceutical composition can be prepared, for example, by combining the desired amounts of the one or more active pharmaceutical ingredients, the selected bacterial biofilm inhibitor (for example, xylitol), and the selected mucosal lubricant and mixing the components by trituration until a uniform liquid is formed. The resulting uniform liquid is then transferred to an Unguator® jar (Gako, Germany) and combined with an aqueous solution of the selected in situ gelling agent (for example, 20% w/v solution of Polaxamer 407) at an amount to bring the total amount of the mixture to the desired total amount. The resulting mixture is then mixed thoroughly with an electric motor and pestle machine at a medium setting for approximately 2 minutes. The Unguator® jar (Gako, Germany) containing the resulting mixture is then stored in a cold environment (about 5° C.) for approximately 14 hours in order for the mixture to set and to release unwanted air pockets. The mixture is then transferred from the Unguator® jar to a sterile, autoclave compatible vial and is then completely sterilized in an autoclave and stored under cool conditions. Under sterile conditions, the cool mixture is then transferred to 10 mL sterile luer/slip syringes and capped with sterile Luer/Slip tips and then stored under cool conditions until ready for administration.

In embodiments where one of more active pharmaceutical ingredients are non-soluble or poorly water soluble, the pharmaceutical composition can be prepared, for example, by combining the one or more active pharmaceutical ingredient, the biofilm inhibiting agent (e.g. xylitol) and a mucosal lubricant comprising caprylic triglyerides and mixing these components by trituration to form a uniform liquid. The resulting uniform liquid is then transferred to an Unguator® jar (Gako, Germany) and combined with an aqueous solution of the selected in situ gelling agent (for example, 20% w/v solution of Polaxamer 407) at an amount to bring the total amount of the mixture to the desired amount. The resulting mixture is then thoroughly mixed in the Unguator® jar (Gako, Germany) by an electric mortar and pestle for 2 minutes on a medium setting. The Unguator® jar containing the resulting mixture is then stored in a cold environment (5° C.) for approximately 14 hours in order for the mixture to set and to release unwanted air pockets. The mixture is then transferred from the Unguator® jar to a sterile, autoclave compatible vial and is then completely sterilized in an autoclave and stored under cool conditions. Under sterile conditions, the cool mixture is then transferred to 10 mL sterile luer/slip syringes and capped with sterile luer/slip tips and then stored under cool conditions until ready for administration.

### Methods of Administration and Treatment

In a further aspect, disclosed is a method for delivering of one or more active pharmaceutical ingredients to at least one paranasal sinus of a subject in need of treatment. The one or more active pharmaceutical ingredients can be delivered as a pharmaceutical composition as described herein. The one or more active pharmaceutical ingredients can be incorporated into the carrier composition to provide a liquid formulation that can be administered into the at least one paranasal sinus of the subject, wherein upon contact of the liquid formulation with the mucosa of the at least one paranasal sinus, the liquid formulation forms an in situ gel. The liquid formulation can be administered into at least one paranasal sinus using a syringe connected to a vacuum cannula that is capable of being inserted into the subject's sinus cavity.

In a still further aspect, provided is a method for treating a subject suffering from chronic rhinosinusitis. The method comprises the local administration to the paranasal sinuses of the subject to be treated, a therapeutically effective amount of a pharmaceutical composition comprising one or more active pharmaceutical ingredients incorporated into the carrier composition described herein. The pharmaceutical composition can be administered using a syringe connected to vacuum cannula as described above.

As used herein, “therapeutically effective amount” refers to administration of an amount of a given pharmaceutical active, to a subject in need thereof that achieves the desired therapeutic effect. The dose administered can be adjusted according to age, weight and condition of the subject.

In a preferred embodiment, the locally administered pharmaceutical composition for treating chronic rhinosinusitis comprises one or more antibiotics, antifungals, corticosteroids or combinations thereof, including any of the exemplary antibiotics, antifungals, and corticosteroids and preferred concentrations described above. The pharmaceutical composition may further include additional active pharmaceutical ingredients including, but not limited to: antihistamines, decongestants, anaesthetics, prostaglandins, vitamins or combinations thereof. It will be appreciated that the specific choice of active pharmaceutical ingredients will depend on the susceptibility of the pathogens present in paranasal sinuses of the subject to be treated. Other considerations in the selection of the active pharmaceutical ingredients would be evident to the skilled person, including interactions between ingredients and patient allergies.

Although the invention has been described with reference to illustrative embodiments, it is to be understood that the invention is not limited to these precise embodiments, and that various changes and modifications are to be intended to be encompassed in the appended claims. The following examples further illustrate the invention are not intended to limit the invention in any manner.

### EXAMPLES

These examples are described for the purposes of illustration and are not intended to limit the scope of the invention.

In the following examples, the suppliers of the components of the disclosed formulations are PCCA® (London, Ontario), GlaxoSmithKline (Mississauga, Ontario) and Sterimax Inc. (Oakville, Ontario).

### Example 1

The patient was a 78 year old female with an 8 year history of chronic rhinosinusitis with daily and recurrent symptoms including facial congestion, facial pain and pressure, headaches and mucopurulent nasal drainage. The patient achieved limited success with previous treatments which included oral antibiotics, oral/nasal corticosteroids, antibiotic irrigation, saline rinses and suctioning. A positive bacterial culture indicated the presence of multi-drug resistant P. aeruginosa, susceptible only to colistimethate. Colistimethate was only commercially available as a solution for intravenous infusion, requiring hospitalization and constant monitoring for neurotoxicity and nephrotoxicity. In order to avoid the intravenous route of administration and the necessary hospitalization, a novel topical formulation was prescribed, consisting of:

| | | |
|---|---|---|
| Sterimax Inc. Colistimethate | 150 | mg |
| PCCA ® Budesonide USP | 2 | mg |
| PCCA ® Caprylic Triglycerides | 0.5 | mL |
| PCCA ® Xylitol NF | 0.1 | g |

| | |
|---|---|
| PCCA ® Poloxamer 407 NF Gel 20% (w/v) | q.s. to 10 mL |

The formulation was prepared under sterile conditions in accordance with USP 797 Guidelines.

The PCCA® Budesonide USP, PCCA® Caprylic Triglycerides and PCCA® Xylitol NF were combined by trituration to form a uniform liquid. The liquid was then transferred to an Unguator® jar and combined with the PCCA® Poloxamer 407 NF Gel 20% in an amount to bring the total volume of the mixture to 10 mL. The resulting mixture was mixed for 2 minutes in an Unguator® Jar using an electric mortar and pestle machine at a medium setting. The Unguator® Jar containing the mixture was stored in a cold environment (5° C.) for 14 hours in order for the mixture to set and to release unwanted air pockets. The mixture was then transferred to a sterile, autoclave compatible vial and is then completely sterilized in an autoclave and stored under cool conditions. Under sterile conditions, the cool mixture is then combined with Sterimax Inc Colisimethate and transferred to 10 mL sterile luer/slip syringe and capped with a sterile luer/slip tip and then stored under cool conditions until needed for administration.

The patient was treated by administering 10 mL of the resulting formulation into the paranasal sinuses via suction cannula. On re-examination 2 weeks post-treatment, the patient's symptoms had completely resolved and a bacterial culture showed the absence of any bacterial pathogens. Current treatment protocols advise that management of chronic rhinosinusitis is a long-term, intensive effort which may or may not provide symptom resolution. As well, with the patient's long-term and persistent history with chronic rhinosinusitis, and multiple previous treatment failures, the resolution of symptoms in this patient was a surprising and unexpected result.

### Example 2

The patient was a 47 year old female with a 10 year history of chronic rhinosinusitis with symptoms including headaches and mucopurulent discharge. Previous treatment resulted in limited success and included oral antibiotics, oral/nasal corticosteroids, antibiotic irrigation and suctioning. Due to multiple, previous treatment failures, a novel topical formulation was prescribed, consisting of:

| | | |
|---|---|---|
| PCCA ® Doxycycline Hyclate USP | 100 | mg |
| PCCA ® Budesonide USP | 2 | mg |
| PCCA ® Caprylic Triglycerides | 0.5 | mL |
| PCCA ® Xylitol NF | 0.1 | g |

| | |
|---|---|
| PCCA ® Poloxamer 407 NF Gel 20% (w/v) | q.s. to 10 mL |

The formulation was prepared under sterile conditions in accordance with USP 797 Guidelines.

The PCCA® Doxycycline Hyclate USP, PCCA® Budesonide USP, PCCA® Caprylic Triglycerides and PCCA® Xylitol NF were combined by trituration to form a uniform liquid. The liquid was then transferred to an Unguator® jar and combined with the PCCA® Poloxamer 407 NF Gel 20% in an amount to bring the total volume of the mixture to 10 mL. The resulting mixture was mixed for 2 minutes in an Unguator® Jar using an electric mortar and pestle machine at a medium setting. The Unguator® Jar containing the mixture was stored in a cold environment (5° C.) for 14 hours in order for the mixture to set and to release unwanted air pockets. The mixture was then transferred to a sterile, autoclave compatible vial and is then completely sterilized in an autoclave and stored under cool conditions. Under sterile conditions, the cool mixture is then transferred to a 10 mL sterile luer/slip syringe and capped with a sterile luer/slip tip and then stored under cool conditions until needed for administration.

The patient was treated by administering 10 mL of the resulting formulation into the paranasal sinuses via suction cannula. At re-examination 4 weeks following administration of the formulation, the patient was symptom-free. A subsequent 10 mL of formulation (produced in accordance with the method described above) was administered to the patient for prophylaxis. Again, at re-examination 4 weeks later, the patient remained symptom free. Current treatment protocols advise that management of chronic rhinosinusitis is a long-term, intensive effort which may or may not provide symptom resolution. As well, with the patient's long-term and persistent history with chronic rhinosinusitis, and multiple previous treatment failures, the resolution of symptoms in this patient was a surprising and unexpected result.

### Example 3

The patient was a 36 year old female with a 20 year history of chronic rhinosinusitis with symptoms including sinus congestion, mucopurulent drainage, facial pain, migraines. A positive culture indicated the presence of the fungal pathogens C. albicans and C. glabrata. White raised lesions were present as a result of the fungal infection. Treatment with intravenous infusion of amphotericin B was indicated but required hospitalization and was unsuitable for the patient who suffered from various co-morbidities including common variable immunodeficiency and IgG subclass deficiency. To avoid hospitalization, its potential complications and the potential adverse effects of intravenous amphotericin B, a novel topical formulation was prescribed, consisting of:

| | | |
|---|---|---|
| Bristol Myers Squibb ® Fungizone ® (amphotericin | 10 | mg |
| B) Inj. | | |
| PCCA ® Caprylic Triglycerides | 0.5 | mL |
| PCCA ® Xylitol NF | 0.1 | g |

| | |
|---|---|
| PCCA ® Poloxamer 407 NF Gel 20% (w/v) | q.s. to 10 mL |

The formulation was prepared under sterile conditions in accordance with USP 797 Guidelines.

PCCA® Caprylic Triglycerides and PCCA® Xylitol NF were combined by trituration to form a uniform liquid. The liquid was then transferred to an Unguator® jar and combined with the PCCA® Poloxamer 407 NF Gel 20% in an amount to bring the total volume of the mixture to 10 mL. The resulting mixture was mixed for 2 minutes in an Unguator® Jar using an electric mortar and pestle machine at a medium setting. The Unguator® Jar containing the mixture was stored in a cold environment (5° C.) for 14 hours in order for the mixture to set and to release unwanted air pockets. The mixture was then transferred to a sterile, autoclave compatible vial and then completely sterilized in an autoclave and stored under cool conditions. Under sterile conditions, the cool mixture is then combined with Bristol Myers Squibb® Fungizone and transferred to 10 mL a sterile syringe and capped and then stored under cool conditions needed for administration.

The patient was treated by administering 10 mL of the resulting formulation into the paranasal sinuses via suction cannula on three separate occasions (day 1, day 4 and day 9). 3 days after administration of the first dose, the patient experienced a reduction in symptoms. 14 days after the first dose, approximately 75% of the lesions/ulcers from the sinus infection had resolved. Current treatment protocols advise that management of chronic rhinosinusitis is a long-term, intensive effort which may or may not provide symptom resolution. As well, with the patient's long-term and persistent history with chronic rhinosinusitis, and multiple previous treatment failures, the significant reduction of symptoms in this patient was a surprising and unexpected result.

### Example 4

The patient was a 45 year old female with a 4 year history of chronic rhinosinusitis with symptoms including headaches, facial pressure, facial pain, and mucopurulent nasal discharge. The patient had experienced limited success with previous treatments, which included oral antibiotics, topical corticosteroids, irrigation and suctioning. As a result of multiple, previous treatment failures, a novel topical formulation was prescribed, consisting of:

| | | |
|---|---|---|
| Glaxosmithkline ® Ceftazidime Inj (Fortaz ®) | 50 | mg |
| PCCA ® Caprylic Triglycerides | 0.5 | mL |
| PCCA ® Xylitol NF | 0.1 | g |

| | |
|---|---|
| PCCA ® Poloxamer 407 NF Gel 20% (w/v) | q.s. to 10 mL |

The formulation was prepared under sterile conditions in accordance with USP 797 Guidelines.

PCCA® Caprylic Triglycerides and PCCA® Xylitol NF were combined by trituration to form a uniform liquid. The liquid was then transferred to an Unguator® jar and combined with the PCCA® Poloxamer 407 NF Gel 20% in an amount to bring the total volume of the mixture to 10 mL. The resulting mixture was mixed for 2 minutes in an Unguator® Jar using an electric mortar and pestle machine at a medium setting. The Unguator® Jar containing the mixture was stored in a cold environment (5° C.) for 14 hours in order for the mixture to set and to release unwanted air pockets. The mixture was then transferred to a sterile, autoclave compatible vial and is then completely sterilized in an autoclave and stored under cool conditions. Under sterile conditions, the cool mixture is then combined with GSK® Ceftazidime inj. and transferred to 10 mL sterile syringe and capped and then stored under cool conditions until needed for administration.

The patient was treated by administering 10 mL of the resulting formulation into the paranasal sinuses by suction cannula. On re-examination 4 weeks post-treatment, the patient was symptom-free. The patient had not been symptom-free for the previous 4 years. On re-examination after a further 4 weeks, the patient continued to be symptom-free. Current treatment protocols advise that management of chronic rhinosinusitis is a long-term, intensive effort which may or may not provide symptom resolution. As well, with the patient's long-term and persistent history with chronic rhinosinusitis, and multiple previous treatment failures, the resolution of symptoms in this patient was a surprising and unexpected result.

### Example 5

The patient was a 63 year old female with a 9 year history of chronic rhinosinusitis with symptoms including facial swelling/pressure/pain, mucopurulent nasal discharge, and headaches. The patient experienced limited success with previous treatments which included oral antibiotics, oral antifungals, nasal irrigation, topical/oral corticosteroids, manuka honey rinses and photodynamic therapy (methylene blue light). As a result of multiple, previous treatment failures, a novel topical formulation was prescribed, consisting of:

| | | |
|---|---|---|
| PCCA ® Gentamicin Sulfate USP | 160 | mg |
| PCCA ® Budesonide USP | 2 | mg |
| PCCA ® Caprylic Triglycerides | 0.5 | mL |
| PCCA ® Xylitol NF | 0.1 | g |

| | |
|---|---|
| PCCA ® Poloxamer 407 NF Gel 20% (w/v) | q.s. to 10 mL |

The PCCA® Gentamicin Sulfate USP, PCCA® Budesonide USP, PCCA® Caprylic Triglycerides and PCCA® Xylitol NF were combined by trituration to form a uniform liquid. The liquid was then transferred to an Unguator® jar and combined with the PCCA® Poloxamer 407 NF Gel 20% in an amount to bring the total volume of the mixture to 10 mL. The resulting mixture was mixed for 2 minutes in an Unguator® Jar using an electric mortar and pestle machine at a medium setting. The Unguator® Jar containing the mixture was stored in a cold environment (5° C.) for 14 hours in order for the mixture to set and to release unwanted air pockets. The mixture was then transferred to a sterile, autoclave compatible vial and is then completely sterilized in an autoclave and stored under cool conditions. Under sterile conditions, the cool mixture is then transferred to a 10 mL sterile luer/slip syringe and capped with a sterile Luer/Slip tip and then stored under cool conditions until needed for administration.

The patient was treated by administering 10 mL of the resulting formulation into the paranasal sinuses via suction cannula. On re-examination at 2 and 4 weeks post-treatment the patient was symptom free. Current treatment protocols advise that management of chronic rhinosinusitis is a long-term, intensive effort which may or may not provide symptom resolution. As well, with the patient's long-term and persistent history with chronic rhinosinusitis, and multiple previous treatment failures, the resolution of symptoms in this patient was a surprising and unexpected result.

### Example 6

The patient was a 56 year old male with a 4 year history of chronic rhinosinusitis with recurrent sinus infections and symptoms including headaches, facial pressure, mucopurulent discharge, nasal drainage and documented inflammation of nasal mucosa and paranasal sinuses. The patient presented with evidence of bacterial and fungal colonization as confirmed through cultures. The patient had been treated routinely with oral antibiotics and oral antifungals, nasal irrigations and topical/oral corticosteroids, surgery with complications, and visited his otolaryngologist every six weeks. Due to multiple previous treatment failures, a novel topical formulation was prescribed, comprising:

| | | |
|---|---|---|
| PCCA ® Gentamicin Sulfate USP | 160 | mg |
| PCCA ® Itraconzaole EP | 200 | mg |
| PCCA ® Budesonide USP | 2 | mg |
| PCCA ® Caprylic Triglycerides | 0.5 | mL |
| PCCA ® Xylitol NF | 0.1 | g |

| | |
|---|---|
| PCCA ® Poloxamer 407 NF Gel 20% (w/v) | q.s. to 10 mL |

The PCCA® Itraconazole was first combined with the PCCA® caprylic triglycerides until mixed uniformly. The PCCA® Gentamicin Sulfate USP, PCCA® Budesonide USP, and PCCA® Xylitol NF were added to the first mixture and combined by trituration to form a uniform liquid. The liquid was then transferred to an Unguator® jar and combined with the PCCA® Poloxamer 407 NF Gel 20 in an amount to bring the total volume of the mixture to 10 mL. The resulting mixture was mixed for 2 minutes in an Unguator® Jar using an electric mortar and pestle machine at a medium setting. The Unguator® Jar containing the mixture was stored in a cold environment (5° C.) for 14 hours in order for the mixture to set and to release unwanted air pockets. The mixture was then transferred to a sterile, autoclave compatible vial and is then completely sterilized in an autoclave and stored under cool conditions. Under sterile conditions, the cool mixture is then transferred to a 10 mL sterile luer/slip syringe and capped with a sterile Luer/Slip tip and then stored under cool conditions until needed for administration.

The patient was treated by administering 10 mL of the resulting formulation into the paranasal sinuses via suction cannula. At a 2 week follow up by a pharmacist, the patient reported a complete resolution of symptoms, and at a 6 week follow up by an otolaryngologist, a visual inspection showed no evidence of infection. Current treatment protocols advise that management of chronic rhinosinusitis is a long-term, intensive effort which may or may not provide symptom resolution. As well, with the patient's long-term and persistent history with chronic rhinosinusitis, and multiple previous treatment failures, the resolution of symptoms in this patient was a surprising and unexpected result. A second 10 mL dose of the formulation (produced in accordance with the method described above) was administered as a preventative measure at the 6 week follow up by Otolaryngologist.

### Example 7

The patient was a 54 year old female with a nine year history of chronic rhinosinusitis. The patient had recurrent symptoms of facial pressure, headaches, mucopurulent discharge, nasal drainage, obstructed sinuses and documented inflammation of nasal mucosa and paranasal sinuses. The patient presented with evidence of bacterial and fungal colonization as confirmed through cultures. The patient had been routinely treated with oral antibiotics and oral antifungals, nasal irrigations, topical/oral steroids, manuka honey rinses and antimicrobial photodynamic therapy (methylene blue light). As well, the patient previously had two sinus surgeries with complications that resulted in subsequent sinus infections. Based on the most recent cultures, a novel topical formulation was prescribed:

| | | |
|---|---|---|
| PCCA ® Sulfamethoxazole EP | 800 | mg |
| PCCA ® Trimethoprim USP | 160 | mg |
| PCCA ® Itraconazole EP | 200 | mg |
| PCCA ® Budesonide USP | 2 | mg |
| PCCA ® Caprylic Triglycerides | 1.5 | mL |
| PCCA ® Xylitol NF | 0.1 | g |

| | |
|---|---|
| PCCA ® Poloxamer 407 NF Gel 20% (w/v) | q.s. to 10 mL |

The PCCA® Sulfamethoxazole EP, PCCA® Trimethoprim USP, PCCA® Itraconazole EP, PCCA® Budensoide USP and PCCA® Xylitol NF were combined with the PCCA® caprylic triglycerides by trituration until uniform mixture. The mixture was then transferred to an Unguator® jar and combined with the PCCA® Poloxamer 407 NF Gel 20% in an amount to bring the total volume of the mixture to 10 mL. The resulting mixture was mixed for 2 minutes in an Unguator® Jar using an electric mortar and pestle machine at a medium setting. The Unguator® Jar containing the mixture was stored in a cold environment (5° C.) for 14 hours in order for the mixture to set and to release unwanted air pockets. The mixture was then transferred to a sterile, autoclave compatible vial and is then completely sterilized in an autoclave and stored under cool conditions. Under sterile conditions, the cool mixture is then transferred to a 10 mL sterile luer/slip syringe and capped with a sterile Luer/Slip tip and then stored under cool conditions until needed for administration.

The patient was treated by administering 10 mL of the resulting formulation into the paranasal sinuses via suction cannula. At follow up six weeks post treatment, the patient noted a 75% reduction in overall symptoms and the physician's exam showed a significant reduction in inflammation and the presence of biofilms and bacterial and fungal colonization. A second 10 mL dose of the formulation (produced in accordance with the method described above) was administered into the paranasal sinuses by suction cannula. At a further six week follow up, the patient noted a complete resolution of symptoms and a physician's exam found no inflammation and no evidence of bacterial or fungal colonization. Current treatment protocols advise that management of chronic rhinosinusitis is a long-term, intensive effort which may or may not provide symptom resolution. As well, with the patient's long-term and persistent history with chronic rhinosinusitis, and multiple previous treatment failures, the resolution of symptoms in this patient was a surprising and unexpected result. A third preventative dose of 10 mL of the formulation (produced in accordance with the method described above) was administered into the paranasal sinuses by suction cannula. Subsequent cultures were negative for the presence of bacterial or fungal pathogens.

1. A carrier composition comprising:
an in situ gelling agent comprising Poloxamer 407;
a biofilm inhibiting agent, and
a mucosal lubricant.

2. The carrier composition of claim 1, wherein the composition comprises about 20% w/v of the Poloxamer 407.

3. The carrier composition of claim 1, wherein the mucosal lubricant comprises caprylic triglycerides.

4. The carrier composition of claim 3, wherein the composition comprises about 5% to about 15% v/v of the caprylic triglycerides.

5. The carrier composition of claim 1, wherein the biofilm inhibiting agent is xylitol.

6. The carrier composition of claim 5, wherein the composition comprises about 1% to about 3% w/v of the xylitol.

7. The carrier composition of claim 1,
wherein the biofilm inhibiting agent is xylitol;
wherein the mucosal lubricant comprises caprylic triglyceride; and
wherein the carrier composition comprises about 20% w/v of the Poloxamer 407, about 5% to about 15% v/v of the caprylic triglycerides and about 1% to about 3% w/v of the xylitol.

8. The carrier composition of claim 5, wherein the carrier composition comprises about 5-15% v/v of the caprylic triglycerides and about 1% w/v of the xylitol.

9. A pharmaceutical composition comprising at least one active pharmaceutical ingredient and a carrier composition, wherein the carrier composition comprises (a) an in situ gelling agent comprising Poloxamer 407; (b) a biofilm inhibiting agent, and (c) a mucosal lubricant.

10. The pharmaceutical composition of claim 9,
wherein the in situ gelling agent comprises about 20% w/v of Poloxamer 407;
wherein the biofilm inhibiting agent is xylitol;
wherein the mucosal lubricant comprises caprylic triglycerides; and
wherein the carrier composition comprises about 20% v/v of the Poloxamer 407, about 5% to about 15% v/v of the caprylic triglycerides and about 1% to about 3% w/v of the xylitol

11. The pharmaceutical composition of claim 9, wherein the carrier composition comprises about 5-15% v/v of the caprylic triglycerides and about 1% w/v of the xylitol.

12. The pharmaceutical composition of claim 9, wherein the active pharmaceutical ingredient is an antibiotic, an antifungal, an antihistamine, a corticosteroid, a decongestant, an anesthetic, a prostaglandin, or a vitamin.

13. The pharmaceutical composition of claim 9, wherein the active pharmaceutical ingredient is:
an aminoglycoside, a carbapenem, a cephalosporin, a fluoroquinolone, a dihydrofolate reductase inhibitor, a glycopeptide, a penicillin, a polymyxin, a sulfonamide, a tetracycline or the pharmaceutically acceptable salts or esters thereof;
itraconazole, amphotericin B, ketoconazole, fluconazole, micafungin, capsofungin or the pharmaceutically acceptable salts or esters thereof;
diephenhydramine or its pharmaceutically acceptable salts or esters thereof;
lidocaine, ketamine or the pharmaceutically acceptable salts or esters thereof;
budesonide, beclomethasone, ciclesonide, fluticasone, triamcinolone or the pharmaceutically acceptable salts or esters thereof;
misoprostol or its pharmaceutically acceptable salts or esters;
ergocalciferol, cholecalciferol or the pharmaceutically acceptable salts or esters thereof;
phenytoin or its pharmaceutically acceptable salts or esters;
tranilast or its pharmaceutically acceptable salts or esters;
sodium chromoglycate or its pharmaceutically acceptable salts or esters;
arginine or its pharmaceutically acceptable salts or esters; or
fluorescein or its pharmaceutically acceptable salts or ester.

14. A method for delivering of one or more active pharmaceutical ingredients to at least one paranasal sinus of a subject in need of treatment, the method comprising:
incorporating the at least one active pharmaceutical ingredient into a carrier composition to provide a liquid formulation, wherein the carrier composition comprises (a) an in situ gelling agent comprising Poloxamer 407; (b) a biofilm inhibiting agent, and (c) a mucosal lubricant; and
administering the liquid formulation into the paranasal sinus of the subject;
wherein upon contact of the liquid formulation with the mucosa of the paranasal sinus, the liquid formulation forms an in situ gel.

15. The method of claim 14, wherein the liquid formulation is administered into the paranasal sinus of the subject using a syringe connected to a cannula, the cannula being inserted into a sinus cavity, turbinate and/or meatus.

16. The method of claim 14, wherein the subject is in need of treatment for chronic rhinosinusitis.

17. The method of claim 14,
wherein the in situ gelling agent comprises about 20% w/v of Poloxamer 407;
wherein the biofilm inhibiting agent is xylitol;
wherein the mucosal lubricant comprises caprylic triglycerides; and
wherein the carrier composition comprises about 20% w/v of the Poloxamer 407, about 5% to about 15% v/v of the caprylic triglycerides and about 1% to about 3% w/v of the xylitol.

18. The method of claim 17, wherein the carrier composition comprises about 5-15% w/v of the caprylic triglycerides and about 1% w/v of the xylitol.

19. The method of claim 14, wherein the active pharmaceutical ingredient is an antibiotic, an antifungal, an antihistamine, a corticosteroid, a decongestant, an anesthetic, a prostaglandin, or a vitamin.

20. The method of claim 14, wherein the active pharmaceutical ingredient is:
an aminoglycoside, a carbapenem, a cephalosporin, a fluoroquinolone, a dihydrofolate reductase inhibitor, a glycopeptide, a penicillin, a polymyxin, a sulfonamide, a tetracycline or the pharmaceutically acceptable salts or esters thereof;
itraconazole, amphotericin B, ketoconazole, fluconazole, micafungin, capsofungin or the pharmaceutically acceptable salts or esters thereof;
diephenhydramine or the pharmaceutically acceptable salts or esters thereof;
lidocaine, ketamine or the pharmaceutically acceptable salts or esters thereof;
budesonide, beclomethasone, ciclesonide, fluticasone, triamcinolone or the pharmaceutically acceptable salts or esters thereof;
misoprostol or its pharmaceutically acceptable salts or esters;
ergocalciferol, cholecalciferol or the pharmaceutically acceptable salts or esters thereof;
phenytoin or its pharmaceutically acceptable salts or esters;
tranilast or its pharmaceutically acceptable salts or esters;
sodium chromoglycate or its pharmaceutically acceptable salts or esters thereof
arginine or its pharmaceutically acceptable salts or esters; or
fluorescein or its pharmaceutically acceptable salts or ester.
